(19) United States
(12) Patent Application Publication
(10) Pub. No.: US 2019/0183453 A1
(43) Pub. Date: Jun. 20, 2019

(54) ULTRASOUND IMAGING SYSTEM AND METHOD FOR OBTAINING HEAD PROGRESSION MEASUREMENTS

(71) Applicant: General Electric Company, Schenectady, NY (US)

(72) Inventors: Fiona Schwab, Mauerkirchen (AT); Christian Perrey, Mondsee (AT); Yelena Tsymbalenko, Mequon, WI (US); Jos Stas, Ham (BE)

(21) Appl. No.: 15/843,612

(22) Filed: Dec. 15, 2017

(51) Int. Cl. A61B 8/08; A61B 8/00

(52) U.S. Cl. CPC A61B 8/0866; A61B 8/463; A61B 2090/365; A61B 8/467; A61B 8/5223; A61B 8/5246

(57) ABSTRACT

An ultrasound imaging system and method for obtaining head progression measurements includes accessing a reference image frame acquired along a first scan plane and obtaining a first head progression measurement from the reference image frame. The system and method includes acquiring, with an ultrasound probe, a live image along a second scan plane and displaying the reference image frame superimposed over the live image. The system and method includes selecting an image frame from the live image and obtaining a second head progression measurement from the image frame.

### FIELD OF THE INVENTION

This disclosure relates generally to a method and ultrasound imaging system for obtaining head progression measurements. The method and ultrasound imaging system includes displaying a reference image frame superimposed over a live image.

### BACKGROUND OF THE INVENTION

A head progression measurement obtained from an ultrasound image is used as a reliable method for assessing the progress of labor and fetal head descent for pregnant women. One commonly used head progression measurement is an angle of progression (AOP) measurement. The AOP is the angle formed between a long axis of the pubis symphysis and a line extending tangentially from an inferior edge of the pubic symphysis to a fetal skull. The AOP should increase over time for women who will undergo vaginal delivery.

The AOP, and how it changes over time, is a good indicator of fetal station. The AOP, and its progression, has been shown to be a good parameter for determining the type of delivery that would be most appropriate for a given pregnancy. Thus far, AOP has been found to be useful in the prediction of the following: successful vaginal delivery, the length of the second stage of labor, the chances of a successful induction of labor, and vacuum extraction.

In order for AOP, or any other head progression measurement, to be a reliable method to assess the progress of labor and fetal head descent, it is desirable to use ultrasound images acquired from the same, or nearly the same, scan plane. Using ultrasound images acquired from different scan planes may result in undesirable error in the determination of head progression which, in turn, may contribute to an incorrect clinical determination regarding the type of delivery for a given patient.

For these and other reasons, an improved ultrasound imaging system and method for obtaining head progression measurements is desired.

### BRIEF DESCRIPTION OF THE INVENTION

The above-mentioned shortcomings, disadvantages, and problems are addressed herein which will be understood by reading and understanding the following specification.

In an embodiment, a method of obtaining an head progression measurement using an ultrasound imaging system includes accessing, from a memory, a reference image frame that was acquired along a first scan plane, the reference image frame including a fetal head and an anatomical reference structure. The method includes obtaining a first head progression measurement from the reference image frame, acquiring, with an ultrasound probe, a live image comprising a sequence of image frames along a second scan plane, and displaying both the live image and the reference image frame on the display screen at the same time, where the reference image frame is superimposed over the live image. The method includes comparing the live image to the reference image frame, adjusting a position of the ultrasound probe based on comparing the live image to the reference image frame to align the second scan plane with the first scan plane. The method includes selecting an image frame from the live image after aligning the second scan plane with the first scan plane. The method includes obtaining a second head progression measurement from the image frame and displaying the second head progression measurement on the display screen.

In an embodiment, an ultrasound imaging system includes an ultrasound probe, a memory, an input device, a display screen, and a processor in electronic communication with the memory, the input device, and the display screen. The processor is configured to access a reference image frame from the memory that was acquired along a first scan plane, the reference image frame including an anatomical reference structure and a fetal head. The processor is configured to obtain a first head progression measurement from the reference image frame and, acquire, with the ultrasound probe, a live image comprising a sequence of image frames along a second scan plane. The processor is configured to display both the live image and the reference image frame on the display screen at the same time, where the reference image frame is superimposed over the live image. The processor is configured to obtain a second head progression measurement from an image frame selected from the live image, and display the second head progression measurement on the display screen.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart in accordance with an embodiment;

FIG. 3 is a schematic representation of a screenshot in accordance with an embodiment;

FIG. 4 is a schematic representation of a screenshot in accordance with an embodiment; and

FIG. 5 is a schematic representation of a screenshot in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical, and other changes may be made without departing from the scope of the embodiments. The following detailed description is, therefore, not to be taken as limiting the scope of the invention.

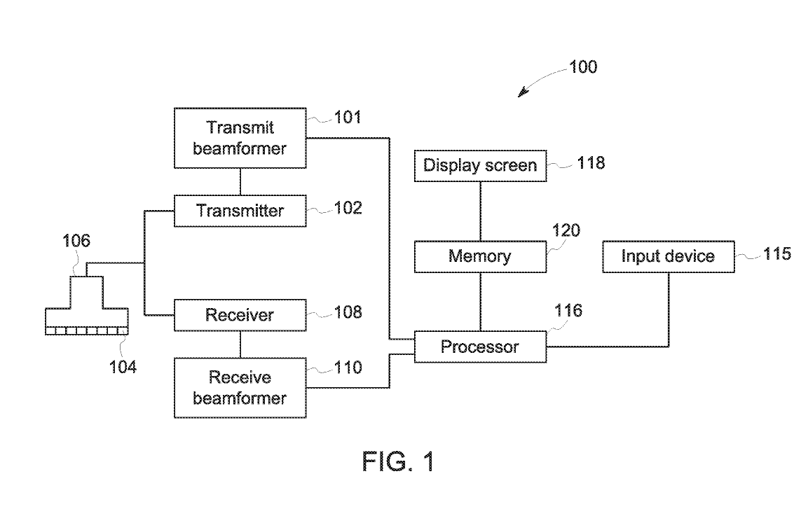

FIG. 1 is a schematic diagram of an ultrasound imaging system 100. The ultrasound imaging system 100 includes a transmit beamformer 101 and a transmitter 102 that drive elements 104 within an ultrasound probe 106 to emit pulsed ultrasonic signals into a body (not shown), The ultrasound probe 106 may be any type of ultrasound probe including a linear probe, a sector probe, a convex prove, and a phased array probe. The ultrasound probe 106 may have the elements 104 arranged in a 1D array, a 1.25D array, a 1.5D array, a 1.75D array, or a 2D array, According to an embodiment, the ultrasound probe 106 may be capable of acquiring real-time 3D ultrasound images. For example, the ultrasound probe 106 may be a mechanical probe that sweeps or oscillates an array in order to acquire the real-time 3D ultrasound data, or the ultrasound probe 106 may be a 2D matrix array with full beam-steering in both the azimuth and elevation directions. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104, and the electrical signals are received by a receiver 108. The electrical signals representing the received echoes are passed through a receive beamformer 110 that outputs ultrasound data. According to some embodiments, the ultrasound probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the ultrasound probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. The ultrasound imaging system 100 includes an input device 115. The input device 115 may be used to control the input of patient data, or to select various modes, operations, and parameters, and the like, The input device 115 may include one or more of a keyboard, a dedicated hard key, a touch pad, a mouse, a track ball, a rotary control, a slider, and the like. The input device may include a proximity sensor configured to detect objects or gestures that are within several centimeters of the proximity sensor. The proximity sensor may be located on either the display screen 118 or as part of a touch screen. The input device 115 may include a touch screen that is positioned in front of the display screen 118. The combination of the touch screen and the display screen results in a touch-sensitive display screen. The touch screen may, for instance, include a smooth front surface made of glass or plastic and a plurality of touch sensors or proximity sensors located beneath the front surface. Each of the touch sensors may, for instance, be a capacitive sensor or a pressure sensor. Each of the proximity sensors may be an electromagnetic field sensor that works by emitting an electromagnetic field and detecting disturbances in the electromagnetic field caused by the proximity of a user's finger or hand, for instance. According to an embodiment, the processor 116 may be configured to display a plurality of user interface icons on the touch-sensitive display screen that may be activated through interactions with the touch screen. For embodiments where the input device 115 is a touch screen, the user interface may include the combination of the touch-sensitive display screen and user interface icons displayed on the display screen 118. The user interface may also include one or more physical controls (such as buttons, sliders, rotary knobs, keyboards, mice, trackballs, etc.) either alone or in combination with graphical user interface icons displayed on the display screen. According to some embodiments, the user interface may include a combination of physical controls (such as buttons, sliders, rotary knobs, keyboards, mice, trackballs, etc.) and user interface icons displayed on either the display screen 118 or on a touch-sensitive display screen. The display screen 118 may be configured to display a graphical user interface (GUI) from instructions stored in a memory 120. The GUI may include user interface icons to represent commands and instructions. The user interface icons of the GUI are configured so that a user may select commands associated with each specific user interface icon in order to initiate various functions controlled by the GUI. For example, various user interface icons may be used to represent windows, menus, buttons, cursors, scroll bars, etc. According to embodiments where the input device 115 includes a touch screen, the touch screen may be configured to interact with the GUI displayed on the display screen 118. The touch screen may be a single-touch touch screen that is configured to detect a single contact point at a time or the touch screen may be a multi-touch touch screen that is configured to detect multiple points of contact at a time. For embodiments where the touch screen is a multi-point touch screen, the touch screen may be configured to detect multi-touch gestures involving contact from two or more of a user's fingers at a time. The touch screen may be a resistive touch screen, a capacitive touch screen, or any other type of touch screen that is configured to receive inputs from a stylus or one or more of a user's fingers. According to other embodiments, the touch screen may be an optical touch screen that uses technology such as infrared light or other frequencies of light to detect one or more points of contact initiated by a user.

According to various embodiments, the input device 115 may include an off-the-shelf consumer electronic device such as a smartphone, a tablet, a laptop, etc. For purposes of this disclosure, the term “off-the-shelf consumer electronic device” is defined to be an electronic device that was designed and developed for general consumer user and one that was not specifically designed for use in a medical environment. According to some embodiments, the consumer electronic device may be physically separate from the rest of the ultrasound imaging system. The consumer electronic device may communicate with the processor 116 thought a wireless protocol, such Wi-Fi, Bluetooth, Wireless Local Area Network (WLAN), near-field communication, etc. According to an embodiment, the consumer electronic device may communicate with the processor 116 through an open Application Programming Interface (API).

The ultrasound imaging system 100 also includes a processor 116 to control the transmit beamformer 101, the transmitter 102, the receiver 108, and the receive beamformer 110. The processor 116 is configured to receive inputs from the input device 115. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations, The receive beamformer 110 may be configured to perform conventional beamforming techniques as well as techniques such as retrospective transmit beamforming (RTB).

The processor 116 is in electronic communication with the ultrasound probe 106. The processor 116 may control the ultrasound probe 106 to acquire ultrasound data. The processor 116 controls which of the elements 104 are active and the shape of a beam emitted from the ultrasound probe 106. The processor 116 is also in electronic communication with the display screen 118, and the processor 116 may process the ultrasound data into images for display on the display screen 118. The processor 116 may be configured to display one or more non-image elements on the display screen 118. The instructions for displaying each of the one or more non-image elements may be stored in the memory 120, which will be described in additional detail hereinafter. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU), or an other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation may be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time volume rates may vary based on the size of the volume from which data is acquired and the specific parameters used during the acquisition. The data may be stored temporarily in a buffer during a scanning session and processed in less than real-time in a live or off-line operation. Some embodiments of the invention may include multiple processors(not shown) to handle the processing tasks. For example, an embodiment may use a first processor to demodulate and decimate the RF signal and a second processor to further process the data prior to displaying an image. It should be appreciated that other embodiments may use a different arrangement of processors. For embodiments where the receive beamformer 110 is a software beamformer, the processing functions attributed to the processor 116 and the software beamformer hereinabove may be performed by a single processor, such as the receive beamformer 110 or the processor 116. Or the processing functions attributed to the processor 116 and the software beamformer may be allocated in a different manner between any number of separate processing components.

According to an embodiment, the ultrasound imaging system 100 may continuously acquire real-time 3D ultrasound data at a volume-rate of, for example, 10 Hz to 30 Hz. A live image may be generated based on the real-time 3D ultrasound data. The live image may be refreshed at a frame-rate that is similar to the volume-rate. Other embodiments may acquire data and or display the live image at different volume-rates and/or frame-rates. For example, some embodiments may acquire real-time 3D ultrasound data at a frame-rate of less than 10 Hz or greater than 30 Hz depending on the size of the volume and the intended application. Other embodiments may use 3D ultrasound data that is not real-time 3D ultrasound data. The memory 120 is included for storing processed frames of acquired data and instructions for displaying one or more non-image elements on the display screen 118. In an exemplary embodiment, the memory 120 is of sufficient capacity to store image frames of ultrasound data acquired over a period of time at least several seconds in length. The memory 120 may comprise any known data storage medium. In embodiments where the 3D ultrasound data is not real-time 3D ultrasound data, the 3D ultrasound data may be accessed from the memory 120, or any other memory or storage device. The memory or storage device may be a component of the ultrasound imaging system 100, or the memory or storage device may external to the ultrasound imaging system 100.

Optionally, embodiments of the present invention may be implemented utilizing contrast agents and contrast imaging. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate and combinations thereof, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory and displays the image frames in real time while a procedure is being carried out on a patient. A video processor module may store the image frames in an image memory, from which the images are read and displayed.

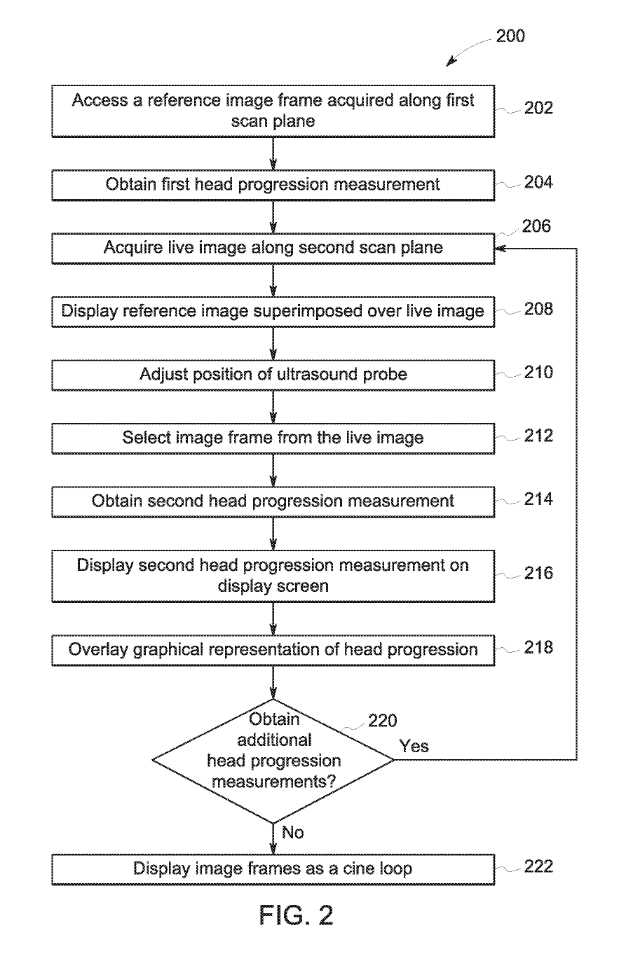

FIG. 2 is a flow chart of a method 200 in accordance with an exemplary embodiment. The individual blocks of the flow chart represent steps that may be performed in accordance with the method 200. Additional embodiments may perform the steps shown in a different sequence, and/or additional embodiments may include additional steps not shown in FIG. 2. The technical effect of the method 200 is the display of a reference image frame superimposed over a live image in order obtain a second head progression measurement from an image frame acquired along a second scan plane that is aligned with a first scan plane.

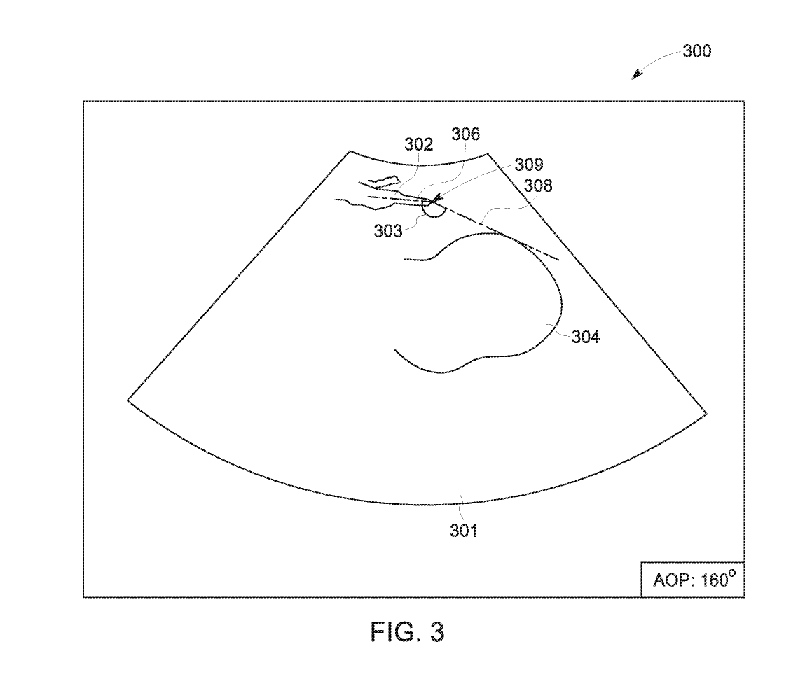

FIG. 3 is a schematic representation of a screenshot 300 in accordance with an embodiment. The screenshot 300 includes a representation of a reference image frame 301 in accordance with an embodiment.

The method 200 will be described with respect to an exemplary embodiment where the method 200 is performed with the ultrasound imaging system 100 shown in FIG. 1. The reference image frame 301, shown in FIG. 3, will also be used to describe the method 200. The method 200 will be described with respect to an exemplary embodiment where the head progression measurement comprises an angle of progression measurement, but it should be appreciated that other embodiments may use a different head progression measurement. The head progression measurement is an objective measurement of the position of the fetal head with respect to the mother's anatomy. For example, the head progression measurement may include a distance between the fetal head and an anatomical reference structure. For example, the distance may be measured between the fetal head and a ramus or the pubic symphysis. Additionally, the head progression measurement may include either a distance of the fetal head from other anatomical reference structures and/or an angle of the fetal head with respect to different anatomical reference structures in other embodiments.

At step 202, the processor 116 accesses a reference image frame 301 acquired along a first scan plane from a memory 120. The reference image frame 301 may have been previously acquired with the ultrasound imaging system 100 or the reference image frame 301 may have been acquired with a different ultrasound imaging system. According to an embodiment, the reference image frame 301 includes both a pubic symphysis 302 and a fetal head 304. The reference image frame 301 may be acquired from a 2D acquisition, or the reference image frame 301 may be a representation of a plane from a 3D acquisition.

At step 204, the processor 116 obtains a first head progression measurement from the reference image frame 301. According to an exemplary embodiment, the first head progression measurement may be a first angle of progression measurement 303. The first angle of progression measurement 303 is obtained by measuring the angle between a longitudinal axis of the pubic symphysis, represented by a pubic symphysis line 306, and a tangent line 308 extending tangentially from an inferior edge of the pubic symphysis 302 to the fetal head 304. According to some embodiments the first angle of progression may be determined semi-automatically. For example, one or both of pubic symphysis line 306 and tangent line 308 may be manually placed by an operator. According to other embodiments, the first angle of progression 303 may be determined automatically and the processor 116 may automatically place both pubic symphysis line 306 and tangent line 308 using image processing techniques, to determine the position of the public symphysis 302 and the fetal head 304. Any type of image processing technique may be used including, thresholding, edge detection, intensity vectors, shape recognition, and the like. Angle of progression is a standard measurement that is well-known by those skilled in the art, so it will not be described in additional detail.

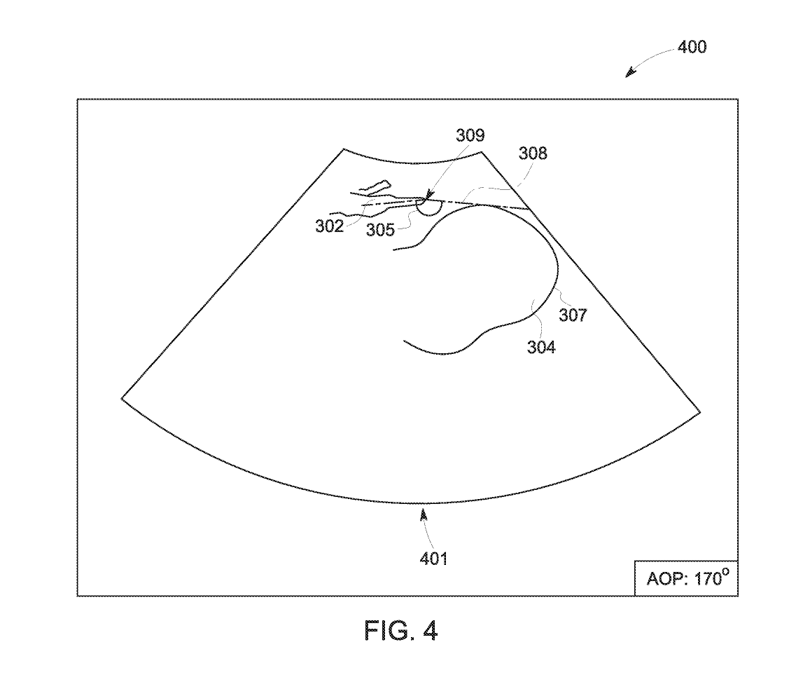

FIG. 4 is a schematic representation of a screenshot 400 in accordance with an embodiment. The screenshot 400 includes a representation of a live image 401 according to an embodiment. At step 206 of the method 200, the operator uses the ultrasound probe 106 to acquire a live image, such as the live image 401, along a second scan plane. The second scan plane may be defined with respect to the ultrasound probe 106. The second scan plane may therefore stay fixed with respect to the ultrasound probe 106, but the position of the second scan plane may move as the position of the ultrasound probe 106 is adjusted with respect to the patient's anatomy. The live image 401 comprises a sequence of image frames acquired along the second scan plane, Image frames in the live image 401 are updated as additional ultrasound data is acquired. At any given point in time, only a single image frame is displayed when viewing the live image 401. According to other embodiments, the live image may include a plane that was acquired as part of live 3D acquisition.

The live image 401 includes the pubic symphysis 302 and the fetal head 304. Since the operator will ultimately want to compare the first angle of progression 303 obtained from the reference image frame 301 with an angle of progression 305 determined from one of the image frames of the live image 401, it is desired to have the second scan plane, represented in the live image 401, align as closely as possible with the first scan plane, represented in the reference image frame 301. Since the live image 401 updates as additional ultrasound data is acquired, it should be appreciated that the position of the second scan plane, as represented in the live image 401, may change during the course of acquiring the live image 401. The operator may, for instance, either intentionally or inadvertently adjust the position of the ultrasound probe 106 with respect to the patient.

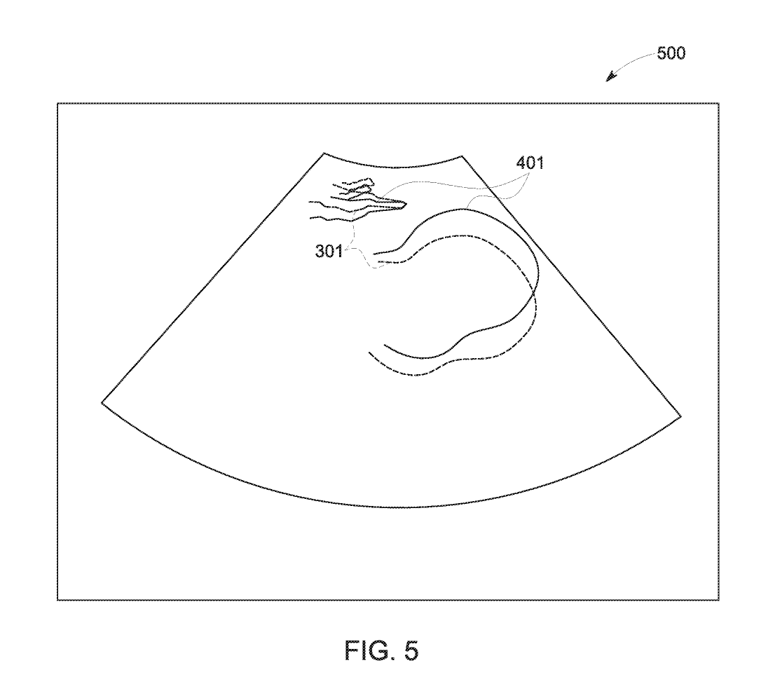

At step 208, the processor displays the reference image frame 301 superimposed over the live image 401. FIG. 5 is a schematic representation of a screenshot 500 in accordance with an embodiment. The screenshot 500 includes a schematic representation of the reference image frame 301, shown in dashed line, superimposed over the live image 401, shown in solid line. While represented in dashed line in FIG. 5, the reference image frame 301 may be displayed differently than the live image 401 to help the user distinguish the reference image frame 301 from the live image 401. For example, the reference image frame 301 may differ in one or more of color, transparency, and intensity from the live image 401. The reference image frame 301 may be displayed with a lower opacity (i.e. a greater transparency) than the live image 401 to help the user distinguish the reference image frame 301 from the live image 401. The reference image frame 301 may be displayed at a lower intensity than the live image 401 to help the user distinguish the reference image frame 301 from the live image 401.

According to an embodiment, the processor 116 may automatically highlight one or more structures in either one or both of the live image 401 and the reference image frame 301 to aid the operator in adjusting the ultrasound probe position so that the live image 401 matches the reference image frame 301. For instance, the processor 116 may use one or more image processing techniques in order to automatically identify the pubic symphysis 302, the fetal head, 304, a portion of the fetal head 304, such as the skull, or shadows present in either one/or both the reference image frame 301 and the live image 401 to help the operator more easily visualize differences between the reference image frame 301 and the live image 401. The operator may use real-time feedback from the live image 401 with respect to the superimposed reference image frame to make adjustments to the position of the ultrasound probe 106. According to some embodiments, the processor 116 may only superimpose select features from the reference image frame 301 on the live image 401. For example, the processor 116 may only superimpose one or more of the pubic symphysis 302, the fetal head 304, or shadows present in the reference image frame 301. At step 210, the operator deliberately adjusts the position of the ultrasound probe 106 to align the second scan plane (as represented by, the live image 401) with the first scan plane (as represented by the reference image frame 301). When the live image 401 matches or closely matches with the reference image frame 301, it is a good indication to the user that the second scan plane is aligned with the first scan plane. According to other embodiments, the matching of the live image 401 to the reference image frame 301 may be based primarily upon on one or more anatomical structures present in both the reference image frame 301 and the live image 401. For example, determining how well the live image 401 matches the reference image frame 301 may be based primarily on how well the pubic symphysis in the live image matches the pubic symphysis in the reference image as the pubic symphysis is expected to a fixed landmark throughout labor.

At step 212, after adjusting the position of the ultrasound probe 106 so that the live image 401 matches the reference image frame 301, an image frame is selected from the live image 401. It should be appreciated that it may not always be possible for the live image 401 to perfectly match the reference image frame 301. The operator may objectively determine when the match between the live image 401 and the reference image frame is close enough, or the processor 116 may use a similarity metric, based on any parameter such as grayscale values, shape-based matching, edge detection, or the like, to determine when the similarity metric exceeds a threshold. The threshold may be selectable by the operator or the threshold may be preset on the ultrasound imaging system 100.

According to an embodiment, the processor 116 may automatically select an image frame from the live image 401 once the similarity metric exceeds the threshold. According to other embodiments, an operator may manually select the image frame from the live image 401 based on an input through the input device 115, such as pressing a freeze button. According to other embodiments, the processor 116 may display a graphical indicator on the display screen 118 to indicate the value of the similarity metric between the live image 401 and the reference image frame 301. Graphical indicators may include the use of various colors, shapes, or icons to indicate the similarity metric between the live image 401 and the reference image frame 301. For instance, a first color may be used to indicate when the similarity metric between the live image 401 and the reference image frame 301 exceeds the threshold. A second color may be used to indicate when the similarity metric between the live image 401 and the reference image frame 301 is below the threshold. According to other embodiments, different icons may be used to indicate when the similarity metric is above or below the threshold. According to one exemplary embodiment, the graphical indicator may include a traffic light icon. The traffic light icon may, for instance, show a green light when the similarity metric exceeds a threshold and a red light when the similarity metric is below the threshold. According other embodiments, the traffic light icon may additionally include a yellow light to indicate when the similarity metric is within a predetermined range of the threshold. The operator may select the image frame from the live image 401 by using the graphical indicator as a guide, or, according to other embodiments, the operator may determine when the match between the live image 401 and the reference image frame 301 is acceptable based solely on a visual comparison.

Next, at step 214, a second head progression measurement is obtained from the image frame selected from the live image 401. According to an exemplary embodiment, the second head progression measurement may be a second angle of progression. As described with respect to step 204, obtaining the second angle of progression measurement may either be an automatic process performed by the processor 116 or a semi-automatic process with one or more inputs required by the operator. For example, the operator may manually identify one or more of the following: the pubic symphysis 302, the pubic symphysis line 306, the fetal head 304, a fetal skull 307, or the tangent line 308.

At step 216, the processor 116 displays the second head progression measurement on the display screen 118. According to an exemplary embodiment, the second head progression measurement may be the second angle of progression measurement. The processor may, for instance, display the second angle of progression measurement in degrees on the display screen 118. The second angle of progression measurement is displayed in the lower right-hand corner of FIG. 4, and it is 170 degrees according to an embodiment.

At step 218, the processor 116 overlays a head progression indicator, such as an angle of progression indicator 309, on the display screen 118. According to an embodiment, the angle of progression indicator 309 includes the pubic symphysis line 302 and the tangent line 308. Different head progression indicators may be used in other embodiments, includimg a line to represent a length.

Steps 220 and 222 are optional for the method 200. Not all embodiments will implement steps 220 and 222. At step 220, it is determined if it is desired to obtain additional head progression measurements. According to an exemplary embodiment, it may be desired to obtain additional head progression measurements on a regular basis. For example, embodiments may obtain additional head progression measurements relatively frequently, such as multiple times a minute or multiple times an hour. Or, the method 200 may obtain additional head progression measurements relatively infrequently, such as at an interval of greater than an hour. If it is desired to obtain an additional head progression measurement, the method 200 returns to step 206, and steps 206, 208, 210, 212, 214, 216, 218, and 220 are repeated. Steps 206, 208, 210, 212, 214, 216, 218, and 220 may be repeated as many times as desired by the operator. If it is not desired to obtain additional head progression measurements, the method 200 advances to step 222.

As discussed previously, step 222 is optional. Some embodiments may be completed after either step 218 or step 220. However, according to some embodiments, at step 222, the method 200 displays the reference image frame and one or more image frames selected during each iteration through steps 206, 208, 210, 212, 214, 216, 218, and 220 as a cine loop. Other embodiments may include displaying a cine loop that does not include the reference image frame. For example, the cine loop may consist of image frame acquired only from multiple iterations of steps 206, 208, 210, 212, 214, 216, 218, and 220. According to an embodiment, the angle of progression indicator 309 may be included in the cine loop. The angle of progression indicator 309 may be adjusted to fit the angle of progression represented in each image frame of the cine loop. Displaying the cine loop allows the operator to easily see how the angle of progression measurement changes during labor. Additionally, the angle of progression indicator provides the operator with a clear visual representation of the progression of labor. For example, by focusing on the angle of progression indicator 309 while viewing the cine loop, the operator can easily see how the angle of progression is changing over the course of labor. The cine loop may represent angle of progression measurements acquired over a relatively short time, such as several minutes up to an hour. Or the cine loop may represent angle of progression measurements acquired over a longer period of time, such as over multiple hours. Either way, watching the angle of progression indicator 309 while viewing the cine loop provides the operator with a very easily discernable representation of how labor is progressing and any changes in the angle of progression/descent of the fetal head that have occurred over the time represented in the cine loop. Different types of head progression indicators may be displayed in other embodiments. For example, a line showing the distance between the fetal head and an anatomical reference structure may be shown in other embodiments. The length of the line may be adjusted as the cine loop is played to visually depict the head progression measurement in each frame of the cine loop.

In the exemplary embodiment described with respect to FIGS. 2-5, the head progression measurement is an angle of progression measurement and the anatomical reference structure is a pubic symphysis. It should be appreciated that a different head progression measurement may be used in other embodiments. For example, in another embodiment, the head progression measurement may be a distance between the fetal head and the ramus. For this embodiment, the anatomical structure is the ramus. Likewise, the angle of progression indicator used to indicate the distance between the fetal head and the ramus would be different than the angle of progression indicator used to show angle of progression. Angle of progression and distance between the fetal head and the ramus are just two exemplary embodiments of this invention. It should be appreciated that other embodiments may use different head progression measurements and/or different anatomical reference structures.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims or if they include equivalent structural elements with insubstantial differences from the literal language of the claims.

We claim:

1. A method for obtaining a head progression measurement using an ultrasound imaging system, the method comprising:
accessing, from a memory, a reference image frame that was acquired along a first scan plane, the reference image frame including a fetal head and an anatomical reference structure;
obtaining a first head progression measurement from the reference image frame;
acquiring, with an ultrasound probe, a live image comprising a sequence of image frames along a second scan plane;
displaying both the live image and the reference image frame on the display screen at the same time, where the reference image frame is superimposed over the live image;
comparing the live image to the reference image frame;
adjusting a position of the ultrasound probe based on said comparing the live image to the reference image frame to align the second scan plane with the first scan plane;
selecting an image frame from the live image after aligning the second scan plane with the first scan plane;
obtaining a second head progression measurement from the image frame; and
displaying the second head progression measurement on the display screen.

2. The method of claim 1, where the anatomical reference structure comprises a ramus and where the head progression measurement comprises a distance between the fetal head and the ramus.

3. The method of claim 1, where the anatomical reference structure comprises a pubic symphysis and the head progression measurement comprises an angle of progression measurement; and where the first head progression measurement comprises a first angle of progression measurement and the second head progression measurement comprises a second angle of progression measurement.

4. The method of claim 3, further comprising overlaying an angle of progression indicator on the image frame displayed on the display screen.

5. The method of claim 3, further comprising displaying both the reference image frame and the image frame as part of a cine loop to show the difference between the first angle of progression measurement and the second angle of progression measurement.

6. The method of claim 5, further comprising overlaying an angle of progression indicator on the cine loop, where the angle of progression indicator is adjusted between the reference image frame and the image frame to represent the difference between the first angle of progression measurement and the second angle of progression measurement.

7. The method of claim 1, where selecting the image frame from the live image comprises pressing a freeze button.

8. The method of claim 1, wherein said comparing comprises comparing the live image to the reference image frame with an image processing algorithm.

9. The method of claim 8. wherein selecting the image frame from the live image comprises automatically selecting the image frame when a similarity metric between the live image and the reference image frame exceeds a threshold.

10. The method of claim 1, further comprising displaying a graphical indicator to indicate a similarity metric between the reference image and the live image.

11. The method of claim 3, where obtaining the first angle of progression measurement and obtaining the second angle of progression measurement are both performed automatically by a processor.

12. The method of claim 1, where obtaining the first angle of progression measurement and obtaining the second angle of progression measurement are both performed semi-automatically.

13. The method of claim 1, where the reference image frame is displayed at a lower opacity than the live image when the reference image frame is superimposed on the live image.

14. The method of claim 1, where the reference image frame is displayed in a different color than the live image when the reference image frame is superimposed on the live image.

15. A ultrasound imaging system comprising:
an ultrasound probe;
a memory;
an input device;
a display screen; and
a processor in electronic communication with the memory, the input device, and the display screen;
wherein the processor is configured to:
access a reference image frame from the memory that was acquired along a first scan plane, the reference image frame including an anatomical reference structure and a fetal head;
obtain a first head progression measurement from the reference image frame;
acquire, with the ultrasound probe, a live image comprising a sequence of image frames along a second scan plane;
display both the live image and the reference image frame on the display screen at the same time, where the reference image frame is superimposed over the live image;
obtain a second head progression measurement from an image frame selected from the live image; and
display the second head progression measurement on the display screen.

16. The ultrasound imaging system of claim 15, where the processor is configured to automatically select the image frame from the live image based on a similarity metric comparing the reference image frame to the live image.

17. The ultrasound imaging system of claim 15, where the processor is further configured to display a head progression indicator on the image frame displayed on the display screen.

18. The ultrasound imaging system of claim 15, wherein the processor is further configured to display both the reference image frame and the image frame as part of a cine loop to show the difference between the first head progression measurement and the second head progression measurement.

19. The ultrasound imaging system of claim 18, wherein the processor is further configured to display a head progression indicator on the cine loop, where the head progression indicator is adjusted between the reference image frame and the image frame to represent the difference between the first head progression measurement and the second head progression measurement.

20. The ultrasound imaging system of claim 14, wherein the head progression measurement comprises an angle of progression measurement.
